(19) United States
(12) Patent Application Publication
(10) Pub. No.: US 2012/0046634 A1
(43) Pub. Date: Feb. 23, 2012

(54) Pull-Release and Refastenable Underpant-type Undergarment

(76) Inventor: Anthony Shields, Salt Lake City, UT (US)

(21) Appl. No.: 12/861,739

(22) Filed: Aug. 23, 2010

(51) Int. Cl. A61F 13/15

(52) U.S. Cl. 604/391; 604/396

(57) ABSTRACT

The present invention provides an improved quick-release and refastenable underpants-type undergarment. The front and back panels of the undergarment are attached at the hip with an openable fastener on at least one side of the undergarment, and are also attached at the crotch with an openable fastener. The fastenings can be comprised of any type of mating-pair compatible fastener devices, such as Velcro. The hip and crotch fasteners allow the undergarment to be easily and quickly removed by pulling on the undergarment, regardless of whether the wearer is sitting, prone, mobile or immobile. The undergarment can also be reattached in position on the wearer even if the wearer cannot stand, balance, or move their legs. The embodiment with a single hip opening can be removed, washed, and used in a single piece and does not require reattaching multiple pieces upon re-use.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable.

### BACKGROUND OF THE INVENTION

The present invention is in the field of quick-release and refastenable undergarments. More particularly, the present invention is in the field of quick-release and refastenable underpant-type undergarments that can be quickly removed by pulling on the undergarment, regardless of the wearer's position and without the necessity of full mobility by the wearer, and can similarly be fastened in position without the necessity of standing, bending, or pulling over the legs.

Conventional underpant-type undergarments must be stepped into one leg at a time while in a standing or sitting position and pulled up the legs and into position on the body to properly wear the undergarment. Conventional underpant-type undergarments therefore require the wearer to bend over, balance, stand or lift the hips, and adequately move their legs and pelvis. If the wearer cannot perform these functions, the wearer must have assistance from another person to put on and take off the undergarment, which requires the assistant to be able to move the wearer and bear the wearer's weight. If the wearer is fully or partially immobile or unconscious or laying down in prone position, removal of conventional underpants-type undergarments requires another person to lift the undergarment from underneath the wearer before pulling it down around the wearer's legs, or cutting the undergarment off the wearer's body, thus rendering the undergarment unusable in the future.

The present invention allows a wearer to put on and remove a reusable underpants-type undergarment without the necessity of standing, bending, or bearing weight. In an emergency situation in which a caregiver must remove the undergarment quickly from a wearer who may be partially or totally immobile, the undergarment may be removed quickly by pulling on the item, which will release it from the wearer's body. The same item can be refastened in position on the wearer without the necessity of standing up, and can be re-used in the same manner as any other undergarment. The current invention can be embodied so that it remains in a single piece when it is removed and does not require reassembly or two or more separate parts or pieces. In addition, the current invention can be embodied with various materials, including both absorbable diaper-type materials and a variety of regular non-absorbable materials used for non-diaper type undergarments.

The prior art illustrate various kinds of undergarments, both with and without absorbent lining, with reopenable fastenings. U.S. Pat. No. 4,597,110 discloses underpants with a single crotch opening and fastener. U.S. Pat. No. 5,535,452 discloses an undergarment for men with a refastenable opening in the front of the garment. U.S. Pat. No. 5,903,922 discloses a longjohn type undergarment attached with a hook and loop fastener and slit opening. U.S. Pat. No. 6,092,242 discloses an hourglass-shaped undergarment comprised of two shaped half width segments held together by a releasable fastener joining two overlapping front panel half width segments, with an area fro accepting an absorbent pad insert. U.S. Pat. No. 2,500,255 discloses an undergarment or diaper with front and rear panels secured directly with a fastening and without a waistband. U.S. Pat. No. 3,094,990 discloses an undergarment with attached suspenders on the waistband. U.S. Pat. Nos. 5,423,789 and 5,704,933 both disclose forms of undergarments with fully detachable waist straps, which do not permanently attach to the undergarment panels. U.S. Pat. No. 6,560,786 discloses an undergarment with a flexible strap attaching the front and back panels at the wearer's hips.

### BRIEF SUMMARY OF THE INVENTION

The present invention concerns an underpants-type undergarment with a front panel worn on the front of the wearer's body and a rear panel worn on the rear of the wearer's body, with the garment passing under the crotch to join the front and rear panels. The top corners of the undergarment are also attached, such that when the garment is fully fastened in wearing position, it fits the wearer in typical underpants fashion. The front and rear panels may join directly at the crotch or may be joined by a separate crotch panel that indirectly joins the front and rear panels.

The present invention particularly concerns fastener devices that fasten an opening between the front and back panels on at least one side of the undergarment by the hip of the wearer and also at an opening at the crotch of the undergarment. At both the hip and crotch openings, the undergarment may be quickly opened or fastened with fasteners that are permanently attached to the undergarment. Any type of mating-pair compatible fastener devices may be used, including snap fastener devices and hook and mating lock Velcro type fasteners.

The present invention improves upon the prior art by disclosing an undergarment with permanently attached fasteners connecting openings at both the side of the wearer's body at the hip area and at the crotch of the undergarment, thus allowing for quick release and removal of the undergarment completely from the wearer's body in any position, whether or not the wearer is standing. In addition, in the embodiment with a single hip opening and the crotch opening (rather than a hip opening on both hips), the present invention can be removed and will remain in a single piece, so that it does not require working with multiple parts or reattaching multiple pieces when used again.

### BRIEF DESCRIPTION OF THE SEVERAL VIEWS OF THE DRAWING

FIG. 1 is a top-front, three-quarter view illustrating the present invention in an embodiment with a single side opening on the left side (from the wearer's perspective) and a crotch-opening, with the fasteners at both the left side opening and the crotch opening in closed position, as the invention would appear when being worn by the wearer;

FIG. 2 is the same perspective as FIG. 1, illustrating the present invention in an embodiment with a single side opening on the left side and a crotch opening, with the left side fastener opened and the crotch fastener closed so that the undergarment is partially open;

FIG. 3 is a perspective from the top looking down into the undergarment, illustrating the present invention in an embodiment with a crotch panel in addition to the front panel and back panel, and a single side opening on the left side and a crotch opening, showing both the left-side fastener and the crotch fastener in open position as the undergarment would appear when fully open and in a position to remove the undergarment from the wearer;

FIG. 4 shows the same perspective as FIG. 3, but without the crotch panel;

FIG. 5 shows the same perspective as FIGS. 1 and 2, showing the present invention in an embodiment with two side openings, one on both the right and left sides of the undergarment, and a crotch opening, with the crotch fastener in a closed position and both the right-side fastener and the left-side fastener in the open position.

### DETAILED DESCRIPTION OF THE INVENTION

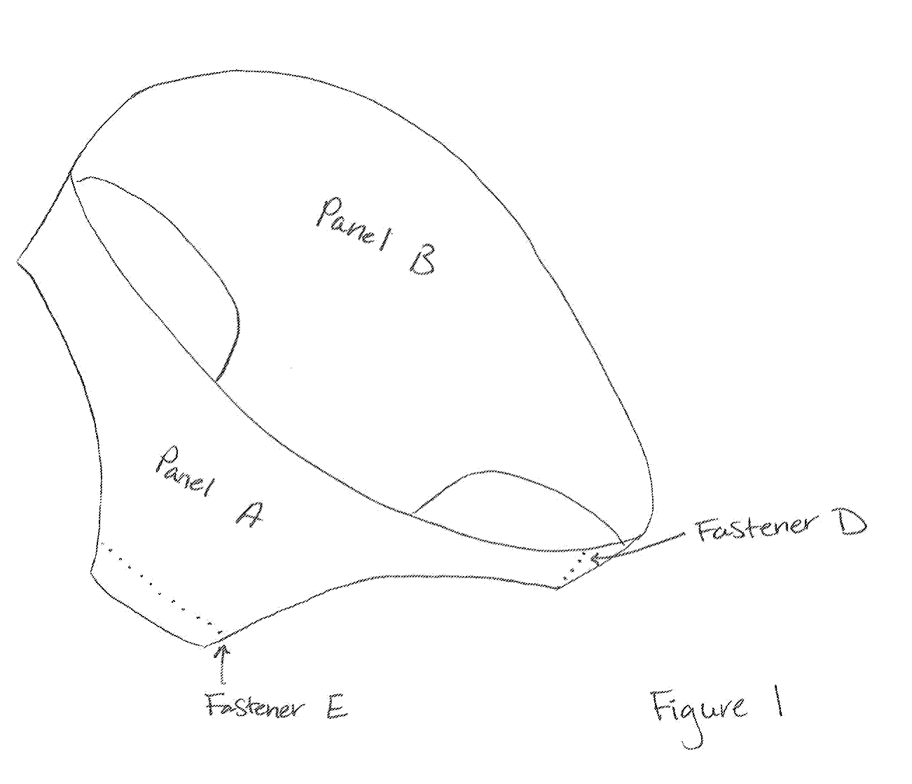

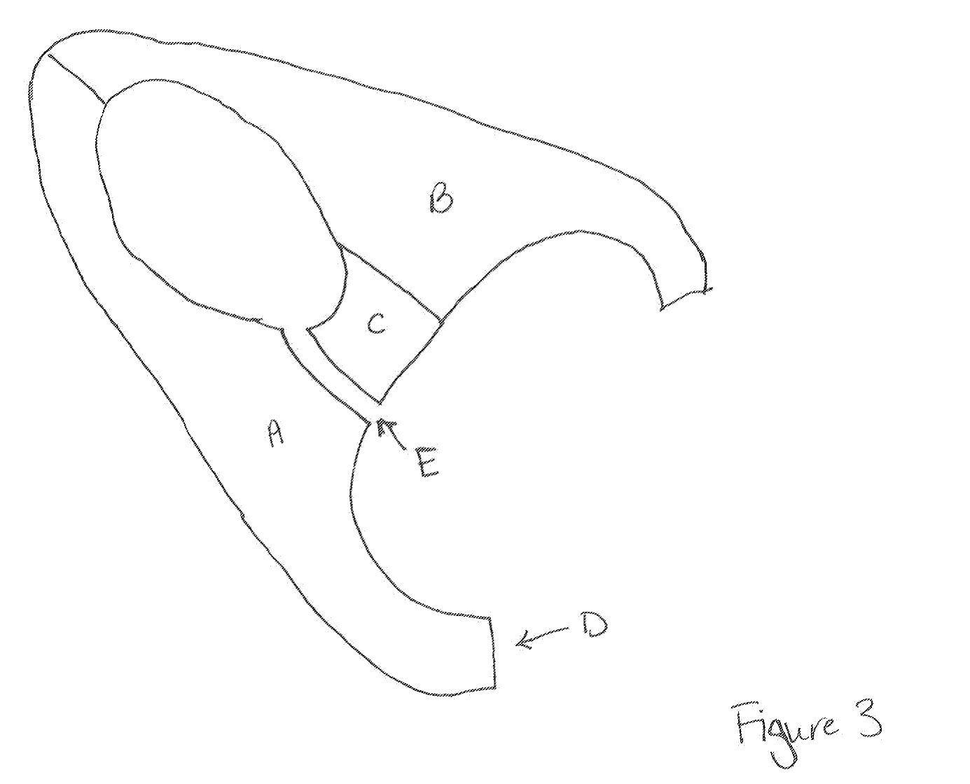

Referring now to the invention in more detail, FIGS. 1-5 show an undergarment that may be embodied with any variety of fastener devices. The undergarment may be comprised of a front panel A and back panel B that are directly attached at the crotch, as in FIGS. 1, 2, 4, and 5, or a front panel A and back panel B that are indirectly attached with a third crotch panel C, as in FIG. 3.

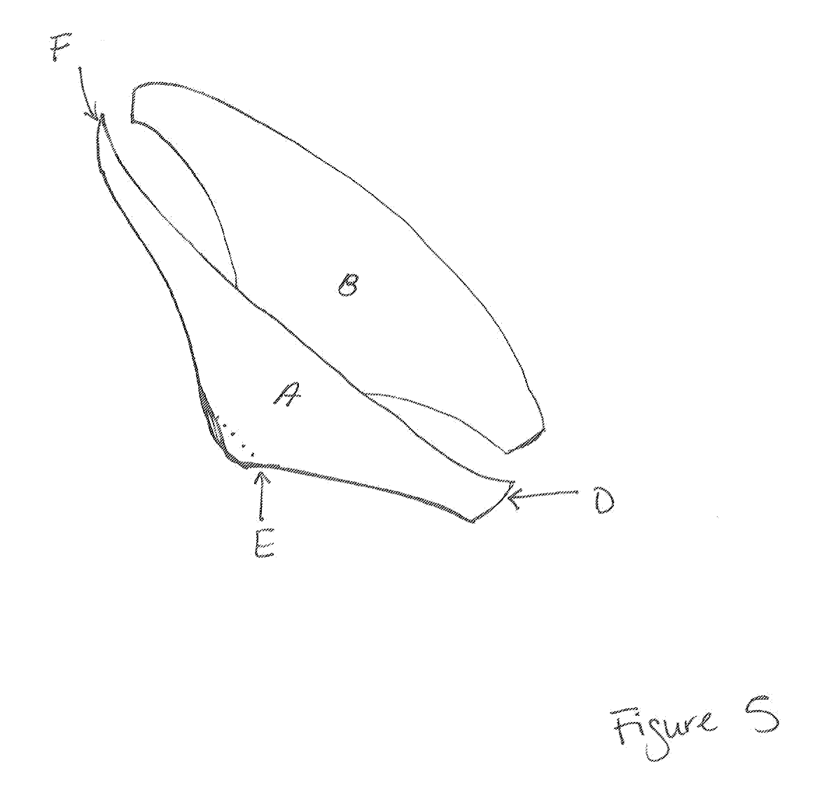

In further detail, FIG. 1 shows an embodiment of the present invention in its completely closed and fastened state, as the invention would appear while being worn by the wearer. In this position, the opening on the left side of the undergarment at the hip region is closed by side fastener D and the crotch opening of the undergarment is closed by crotch fastener E. FIG. 5 shows an embodiment of the undergarment from the same perspective, but with openings at both the right and left sides at the hips. In FIG. 5, left side fastener D and right side fastener F are both in the open position, but crotch fastener E is closed.

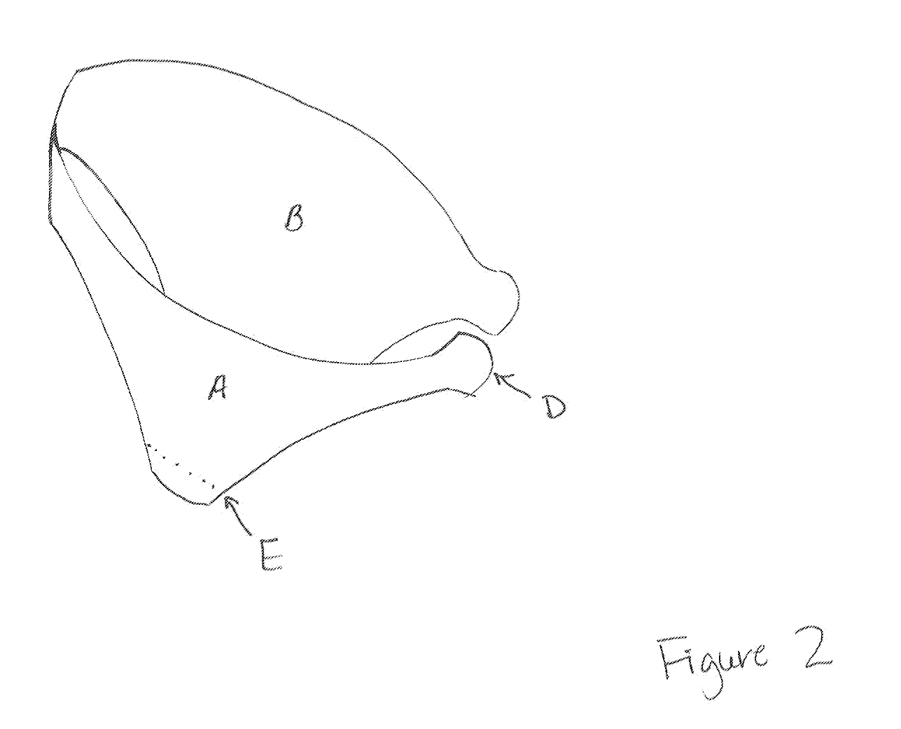

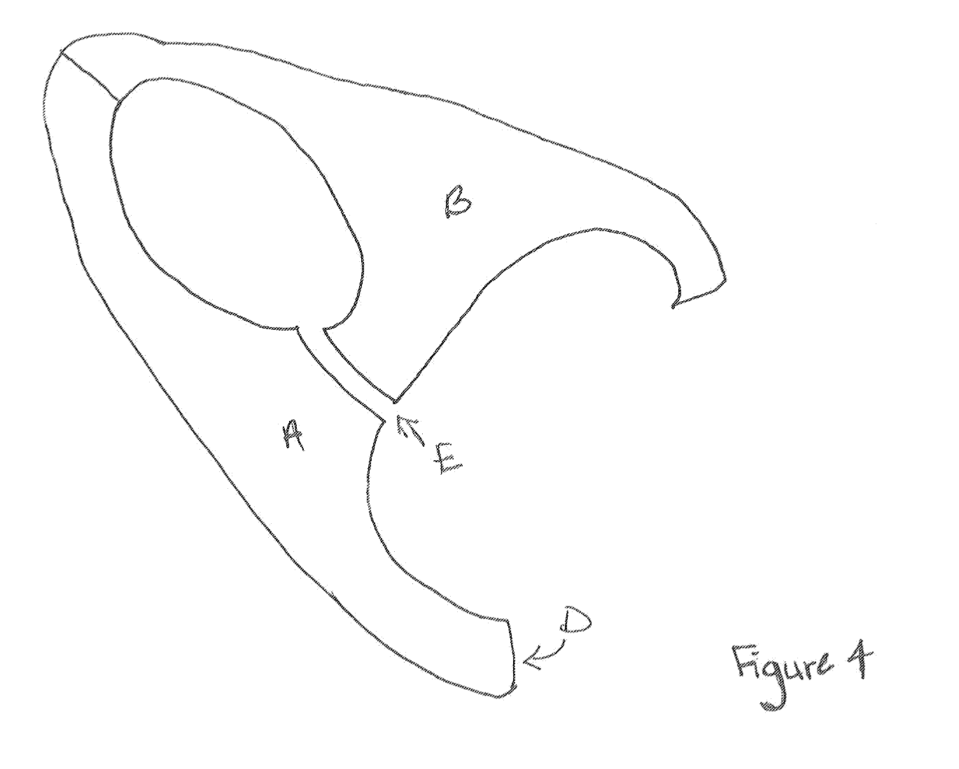

In more detail, FIG. 2 shows an embodiment of the present invention with left side fastener D in open position and crotch fastener E in closed position. FIG. 3 shows the present invention from a perspective looking down into the undergarment from the top, with both left side fastener D and crotch fastener E in open position, which allows the undergarment to be completely removed from the wearer's body by simply pulling on the undergarment, regardless of the position of the wearer or whether or not they are standing or prone, mobile or immobile. FIG. 3 shows an embodiment with a crotch panel C, which connects front panel A and back panel B between the wearer's legs. FIG. 4 shows the same perspective, but in an embodiment without a crotch panel, so that front panel A and back panel B are directly attached.

The combination of side and crotch openings enables the wearer, or a third-party assisting the wearer, to quickly remove the undergarment by pulling the undergarment away from the wearer's body. Similarly, the positioning of the fasteners allows the wearer or a third party assisting the wearer to fasten the undergarment in position on the wearer's body easily and without requiring the wearer to stand or the assistant to lift. The present invention may be assembled and attached on the wearer's body rather than requiring the wearer to pull the item on over the wearer's legs. Any variety of fastening devices can be used to fasten the undergarment at the crotch and at one or both sides of the undergarment, including Velcro fasteners or snap type fasteners.

While the foregoing written description of the invention enables one of ordinary skill to make and use what is considered presently to be the best mode thereof, those of ordinary skill will understand and appreciate the variations, combinations, and equivalents of the specific embodiment, method, and examples herein. The invention should therefore not be limited by the above described embodiment, method, and examples, but by all embodiments and methods within the scope and spirit of the invention as claimed.

1. An underpants-type undergarment comprising: at least two panels including a front panel having a front top edge and extending down the front of a wearer's body from the front top edge, the front panel extending to the crotch of the body, and a rear panel having a rear top edge and extending down the rear of the wearer's body from the rear top edge to the crotch, wherein the front and back panels are shaped so that each has left and right upper corners with respect to the left and right sides of the body at both the front and the rear top edges, and the front and back panels on at least one side of the body being attached to each other with a fastener that opens and closes, each of the front and back panels on that side having a respective portion of the fastening element thereon, so that the undergarment opens at the hip of the wearer's body by opening the hip fastener and closes by closing the hip fastener, and the front and back panels also being attached to each other with a fastener that opens and closes at the crotch, with each of the front and back panels at the crotch having a respective portion of the fastening element thereon, so that the undergarment opens at the crotch of the wearer's body by opening the crotch fastener and closes by closing the crotch fastener.

2. The undergarment of claim 1, further comprising a separate crotch panel at the crotch between the front and the rear panels, and having the crotch fastener and opening claimed in claim 1 occurring at the crotch of the front panel and the front of the crotch panel.

3. The undergarment of claim 1, wherein the hip opening and hip fastener claimed in claim 1 exist on both the right and left sides of the undergarment.

4. The undergarment of claim 2, where the hip opening and hip fastener claimed in claim 2 exist on both the right and left sides of the undergarment.

5. The undergarment of claim 1, wherein the first fastening device at the hip and the second fastening device at the crotch are hook and loop Velcro type fasteners.

6. The undergarment of claim 1, wherein the first fastening device at the hip and the second fastening device at the crotch are snap fasteners.

7. The undergarment of claim 2, wherein the first fastening device at the hip and the second fastening device at the crotch are hook and loop Velcro type fasteners.

8. The undergarment of claim 2, wherein the first fastening device at the hip and the second fastening device at the crotch are snap type fasteners.

9. The undergarment of claim 3, wherein the first fastening device at the right hip, the second fastening device at the left hip, and the third fastening device at the crotch are hook and loop Velcro type fasteners.

10. The undergarment of claim 3, wherein the first fastening device at the right hip, the second fastening device at the left hip, and the third fastening device at the crotch are snap type fasteners.

11. The undergarment of claim 4, wherein the first fastening device at the right hip, the second fastening device at the left hip, and the third fastening device at the crotch are hook and loop Velcro type fasteners.

12. The undergarment of claim 4, wherein the first fastening device at the right hip, the second fastening device at the left hip, and the third fastening device at the crotch are snap type fasteners.
